(19) United States
(12) Patent Application Publication
(10) Pub. No.: US 2007/0025949 A1
(43) Pub. Date: Feb. 1, 2007

(54) Compositions and kit for alleviating signs of ageing

(75) Inventors: Isabelle Hansenne, Westfield, NJ (US); Hani Fares, Somerset, NJ (US); Marc Cornell, Jackson, NJ (US); Sidney Peter Foltis, Nutley, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/192,720

(22) Filed: Jul. 29, 2005

(51) Int. Cl. A61K 8/365; A61K 8/97

(52) U.S. Cl. 424/74; 424/401

(57) ABSTRACT

A cosmetic kit for treating human skin comprising a multi-unit receptacle containing: (a) a unit containing a microdermabrasion composition having at least one type of skin abrading particle; (b) a unit containing a peeling composition having at least one skin peeling agent; (c) a unit containing an anti-ageing composition having at least one anti-ageing ingredient; (d) a unit containing a skin soothing composition having at least one skin soothing ingredient; (e) optionally, a unit containing a maintenance composition; and (f) optionally, a unit containing a photoprotective composition.

### BACKGROUND OF THE INVENTION

Women, indeed even men, currently have a tendency to wish to appear young for as long as possible and they are consequently seeking to alleviate signs of ageing of the skin, which show up in particular as wrinkles and fine lines.

To date, wrinkles and fine lines are treated using cosmetic products containing active principles which act either on the epidermis of the skin or in the dermis.

Active principles (generally in the form of hydroxy acids) may be included in cosmetic or dermatological compositions which increase epidermal cell proliferation and/or which are having a keratolytic effect and/or which cause the upper layer of skin to desquamate or peel off. These compositions may be used by the subject herself or in the salon of a skin specialist (depending on the depth of the peeling). They tend to alleviate the signs of ageing of the skin, i.e. reduce the appearance of wrinkles and fine lines. In addition to these “chemical” desquamating or peeling agents, abrasives, such as aluminum oxide, are known to be used for exfoliating the surface of the skin. This exfoliation process is known as “microdermabrasion” or micropeeling. Microdermabrasion is employed in particular by beauticians or skin specialists who use compositions containing aluminum oxide microcrystals for resurfacing the surface layers of the skin. These crystals are sprayed over the skin using a device which subsequently sucks them up with the abraded skin.

Another facet of the reduction of the appearance of wrinkles and fine lines is the use of compositions containing active agents which promote the synthesis, or prevent the deterioration, of elastic fibers (collagen and elastin) or of glycosaminoglycans which are components of cutaneous tissue.

There remains a need for an effective, rapidly visible, and lasting method for reducing wrinkles and fine lines.

The Applicant has discovered that it is possible to satisfy this need by providing the consumer with a multi-unit cosmetic kit and a protocol employing successive application of several compositions.

Thus, the subject of this invention provides a multi-unit receptacle containing various compositions for successive application of microdermabrasion, skin peeling, skin soothing compositions and, optionally, a maintenance composition. Another subject of the invention is a method of using the various compositions contained in the multi-unit receptacle for treating the skin.

### BRIEF SUMMARY OF THE INVENTION

The present invention relates to a cosmetic kit for treating human skin comprising a multi-unit receptacle containing:
(a) a unit containing a microdermabrasion composition, having at least one type of skin abrading particle;
(b) a unit containing a peeling composition having at least one skin peeling agent;
(c) a unit containing an anti-ageing composition having at least one anti-ageing ingredient;
(d) a unit containing a skin soothing composition having at least one skin soothing agent;
(e) optionally, a unit containing a maintenance composition; and
(f) optionally, a unit containing a photoprotective composition.

Another embodiment of the present invention is a cosmetic kit for treating human skin comprising a multi-unit receptacle containing:
(a) a unit containing a microdermabrasion composition having at least one type of skin abrading particle;
(b) a unit containing a line/wrinkle treating composition comprising a mixture of at least one skin peeling agent and at least one anti-ageing ingredient;
(c) a unit containing a skin soothing composition having at least one soothing agent;
(d) optionally, a unit containing a maintenance composition; and
(e) optionally, a unit containing a photoprotective composition.

Another embodiment of the present invention is a cosmetic kit for treating human skin comprising a multi-unit receptacle containing:
(a) a unit containing a microdermabrasion composition having at least one type of skin abrading particle;
(b) a unit containing a multi-benefit composition having at least one skin soothing ingredient and at least one component chosen from a skin peeling agent and an anti-ageing ingredient;
(c) optionally, a unit containing a maintenance composition; and
(d) optionally, a unit containing a photoprotective composition.

According to yet another embodiment of the present invention, there is provided a method for treating skin, intended to soften cutaneous signs of ageing, involving the steps of:
(a) providing a unit containing a microdermabrasion composition having at least one type of skin abrading particle;
(b) providing a unit containing a skin peeling composition having at least one skin peeling agent;
(c) providing a unit containing an anti-ageing composition having at least one anti-ageing ingredient;
(d) providing a unit containing a skin soothing composition;
(e) providing a unit containing a photoprotective composition;
(f) contacting the skin with the microdermabrasion composition to form abraded skin;
(g) removing the microdermabrasion composition from the abraded skin;
(h) mixing the skin peeling composition with the anti-ageing composition to form a line/wrinkle treating composition;
(i) contacting the skin with the line/wrinkle treating composition;
(j) applying a layer of skin soothing composition over the line/wrinkle treating composition;
(k) allowing the line/wrinkle treating composition and the skin soothing composition to remain on the skin for a predetermined period of time;
(l) removing the line/wrinkle treating composition and the skin soothing composition from the skin; and
(m) applying the photoprotective composition onto the skin.

This method is used for an amount of time sufficient for achieving reduction of fine lines and wrinkles of the targeted area of skin. Preferably, the area of skin on which the above method is carried out is an area of skin of the face, preferably the entire face, except for the outline of the eyes.

### DETAILED DESCRIPTION OF THE INVENTION

Other than in the operating examples, or where otherwise indicated, all numbers expressing quantities of ingredients are to be understood as being modified by the term “about”.

The present invention relates to a cosmetic kit for treating human skin comprising a multi-unit receptacle containing: (a) a unit containing a microdermabrasion composition; (b) a unit containing a peeling composition having at least one skin peeling agent; (c) a unit containing an anti-ageing composition having at least one anti-ageing ingredient; (d) a unit containing a skin soothing composition; (e) optionally, a unit containing a maintenance composition; and (f) optionally, a unit containing a photoprotective composition.

The Microdermabrasion Composition

The microdermabrasion composition contains at least one type of skin abrading particle.

Suitable skin abrading particles for use in the present invention include, but are not limited to, metal oxide particles, pumice, polyethylene, jojoba, diatomaceous earth, silica, sugar, ground fruit kernels and wax beads.

Metal oxide particles may include magnesium oxide or aluminum oxide. These particles have a purity of at least 95%, better still of at least 99%. Their average particle size preferably ranges from 100 to 180 μm. Aluminum oxide is preferred for use in the present invention, in particular in the anhydrous crystalline form (corundum).

Aluminum oxide particles are available as particles calcined at high temperature, until the α-Al₂O₃ crystalline structure of corundum is obtained, and then treated in order to form grains having sharp edges and having a given particle size distribution, the particles preferably having an average particle diameter of between 100 and 180 μm and preferably between 130 and 150 μm. Their distribution is preferably such that none of the particles has a diameter greater than 250 μm. Such particles are available commercially in particular from MarkeTech International under the trade name Dermagrain™. The particles referenced as Dermagrain™ 900 are composed of crystalline α-alumina with a purity of 99.55% having an average particle size diameter of approximately 140 μm, the particles all having a diameter of less than 250 μm. Less than 3% of the particles have a diameter of less than 105 μm. Other particles are available from Industrial Supply under the trade names ARL 100 and ARL 120. They are aluminum oxide particles having an average particle diameter of 120 and 100 μm respectively and a particle size distribution ranging from 75 to 212 μm and from 63 to 180 μm respectively.

In another embodiment, the metal oxide particles used in the composition according to the invention may be magnesium oxide preferably having an average particle size ranging from 100 to 180 μm. An example of particles of this type is sold by MarkeTech International under the trade name Magnaderm™ 100. They are particles having an average particle diameter of approximately 120 μm and a purity of at least 99%.

In another embodiment the particles used in the microdermabrasion composition may be ground up fruit kernel powder, such as apricot, peach or nectarine kernel powder, or diatomaceous earth. These powders are available commercially from companies such as Alban Muller.

The skin abrading particles will be present in an amount sufficient to abrade the targeted area of skin. Preferably, the skin abrading particles may be present in concentrations ranging from 0.5 to 40%, preferably from 10 to 30% and more preferably from 15 to 25% by weight, based on the total weight of the microdermabrasion composition. This composition preferably has a pH of greater than 4 and less than 8, preferably of less or equal to 7 and more preferably of between 5.5 and 7.

The microdermabrasion composition contained in the first unit of the multi-unit receptacle according to the invention may be provided in the form of a lotion, gel, fluid or cream. It may comprise various adjuvants and advantageously may include at least one heterogeneous polysaccharide. This is because the Applicant has demonstrated that these compounds make it possible to reduce the discomfort associated with the use of the abovementioned abrasive metal oxide particles. This heterogeneous polysaccharide can be an alginate or alternatively a heterogeneous polysaccharide comprising at least one fucose unit, comprising in particular fucose, galactose and galacturonic acid units, in particular a linear sequence of α-L-fucose, of α-D-galactose and of galacturonic acid. Such a polysaccharide is available in particular in the form of a 1% solution in water from Solabia under the trade name Fucogel 1000 PP®.

This composition may be applied to the skin by manual massaging with the fingertips or by mechanical massaging using a vibrating device provided with a massaging head equipped with a pad, as disclosed in Application US2001/0046506 or U.S. Pat. No. 6,652,888, for example.

The Skin Peeling Composition

The skin peeling composition contains at least one skin peeling agent, present in an amount sufficient to peel the skin.

Suitable skin peeling agents include, but are not limited to: α-hydroxy acids, such as citric, lactic, glycolic, malic, tartaric or mandelic acid; α-hydroxy acids, such as salicylic acid or its derivatives such as 5-(n-octanoyl)salicylic acid (also known as Capryloyl Salicylic Acid under the CTFA designation) such as 5-(n-octanoyl)salicylic acid (also known as Capryloyl Salicylic Acid under the CTFA designation), examples of which are described in U.S. Pat No. 4,767,750, the entire contents of which is hereby incorporated by reference; urea; aminosulphonic compounds and in particular N-(2-hydroxyethyl)piperazine-N′-2-ethanesulphonic acid (HEPES); and derivatives of 2-oxothiazolidine-4-carboxylic acid (procysteine). Lactic acid, glycolic acid, HEPES and their mixtures are preferred for use in the present invention.

The skin peeling agent may preferably be present in an amount of from at least 3 to 20% by weight, preferably 5 to 15% by weight, and more preferably 5 to 10% by weight, based on the total weight of the skin peeling composition.

According to one embodiment of the invention, the skin peeling composition contains 10% glycolic acid. In another embodiment, the skin peeling composition contains a mixture of 3.5% of glycolic acid, 0.5% of lactic acid and 5% of HEPES, all weights based on the total weight of the skin peeling composition.

The skin peeling composition may be in the form of a lotion, gel, fluid or cream. In an alternative form, it can be applied in the form of a mask or patch or can be impregnated on a wipe.

The Anti-Ageing Composition

The anti-ageing composition contains at least one anti-ageing ingredient, present in an amount sufficient to soften the cutaneous signs of skin ageing.

Suitable anti-ageing ingredients include, but are not limited to:

A. compounds which enhance the synthesis of: (i) collagen such as ascorbic acid; extracts of Centella asiatica; asiaticosides and derivatives; synthetic peptides, such as iamine, biopeptide CL or palmitoyloligopeptide, sold by Sederma; peptides extracted from plants, such as the soybean hydrolysate sold by Coletica under the trade name Phytokine®; and plant hormones, such as auxins and lignans; (ii) elastin such as the extract of Saccharomyces cerevisiae sold by LSN under the trade name Cytovitin®; and the extract of the alga Macrocystis pyrifera sold by Secma under the trade name Kelpadelie®; (iii) glycosaminoglycans such as the product of fermentation of milk by Lactobacillus vulgaris sold by Brooks under the trade name Biomin yogourt®; the extract of the brown alga Padina pavonica sold by Alban Muller under the trade name HSP3®; and the extract of Saccharomyces cerevisiae available in particular from Silab under the trade name Firmalift® or from LSN under the trade name Cytovitin®; (iv) proteoglycans; (v) fibronectin such as the extract of Salina zooplankton sold by Seporga under the trade name GP4G®; the yeast extract available in particular from Alban Müller under the trade name Drieline®; and the palmitoyl pentapeptide sold by Sederma under the trade name Matrixyl®; (vi) laminin;

B. compounds which inhibit the decomposition of: (i) collagen such as retinoids and derivatives, oligopeptides and lipopeptides, lipoamino acids, the malt extract sold by Coletica under the trade name Collalift®; extracts of blueberry or of rosemary; lycopene; isoflavones, their derivatives or the plant extracts comprising them, in particular extracts of soybean, for example sold by Ichimaru Pharcos under the trade name Flavosterone SB®, of red clover, of flax, of kakkon or of sage; (ii) elastin such as the peptide extract of Pisum sativum seeds sold by LSN under the trade name Parelastyl®; heparinoids; and pseudodipeptides, such as {2-[acetyl(3-(trifluoromethyl)phenyl)amino]-3-methylbutyrylamino} acetic acid;

C. skin relaxants, such as alverine and its salts, manganese salts and in particular manganese gluconate, magnesium salts and in particular magnesium gluconate and sulphate, the hexapeptide Argireline R, sold by Lipotec, adenosine, and also sapogenins and natural extracts, in particular of Dioscorea opposita or of Dioscorea villosa (wild yam), comprising them, and also extracts of Boswellia serrata;

D. agents which inhibit the glycation of proteins, such as extracts of plants from the family of the Ericaceae, in particular an extract of blueberry (Vaccinium angustifolium); ergothioneine and its derivatives; and hydroxystilbenes and their derivatives, such as resveratrol and 3,3′, 5,5′-tetrahydroxystilbene;

E. agents which enhance the proliferation of: (i) keratinocytes such as retinoids, including retinol and retinyl palmitate, adenosine, phloroglucinol, the extracts of walnut meal sold by Gattefossé and the extracts of Solanum tuberosum sold by Sederma; (ii) fibroblasts such as plant proteins or polypeptides, plant extracts, in particular of soybean, and plant hormones, such as gibberellins and cytokinins;

F. agents which enhance the differentiation of keratinocytes, such as inorganic materials, including calcium; a lupin peptide extract, such as that sold by Silab under the trade name Structurine®; sodium β-sitosteryl sulphate, such as that sold by Seporga under the trade name Phytocohesine®; a water-soluble maize extract, such as that sold by Solabia under the trade name Phytovityl®; a peptide extract of Voandzeia subterranea, such as that sold by Laboratoires Sérobiologiques under the trade name Filladyn LS 9397®; and lignans, such as secoisolariciresinol.

The anti-ageing ingredient, in general, may preferably be present in an amount of from 0.01 to 99% by weight, based on the total weight of the anti-ageing composition. The precise amount of anti-ageing ingredient to be used will depend on its type. For example, in the event that retinol is used as the anti-ageing ingredient, it may be used in rather small amounts due to its ability to be efficacious in those amounts. On the other hand, if ascorbic acid is employed as the anti-ageing ingredient, it can be present in large amounts if used in powder form. The precise amount of anti-ageing ingredient to be used will thus depend on the type of ingredient chosen, its carrier vehicle, as well as other considerations known, and determinable, by those skilled in the art.

The Skin Soothing Composition

The skin soothing composition contains at least one skin soothing agent present in an amount sufficient to both soothe previously weakened skin and to protect it from further assaults, particularly those from the environment.

The skin soothing composition according to the invention will contain at least one skin soothing agent conventionally used to soothe skin. Examples thereof include, but are not limited to, emollients such as silicones, and hydrating agents such as polyols, i.e. glycerin and/or propylene glycol which may be homogenized together in the presence of an emulsifier. It may also comprise additives such as at least one antioxidant for combating free radicals, such as tocopherol, tocopherol acetate, ascorbic acid and arginine pyrrolidonecarboxylate.

The skin soothing agent will typically be present in the composition in an amount of from 1 to 40% by weight, preferably from 3 to 35% by weight, and more preferably from 5 to 25% by weight, based on the weight of the composition. A particularly preferred skin soothing agent is silicone.

The skin soothing composition is generally used in the form of a cream and may be advantageously dispensed from a flexible tube, a pot or any other suitable type of container.

The Maintenance Composition

For some people, the microdermabrasion and peeling steps may result in excessive skin irritation. It may then be recommended to skip these steps every other day and replace them with the application of a maintenance composition. This maintenance composition contains at least one active chosen from a skin peeling agent and an anti-ageing ingredient wherein either, or both, of which may be present in a concentration lower than that of the skin peeling composition and/or anti-ageing composition. In another embodiment of the invention, the maintenance composition may further contain sunscreens. In a particularly preferred embodiment, the maintenance composition contains 5% by weight, based on the weight of the maintenance composition, of ascorbic acid.

The Photoprotective Composition

It is highly recommended that a photoprotective composition, preferably one having an SPF of at least 15, be applied onto treated skin in order to protect it from the harmful effects of the sun.

Suitable organic photoprotective agents may be chosen from anthranilates; cinnamic acid derivatives; dibenzoylmethane derivatives; salicylic acid derivatives; camphor derivatives; triazine derivatives, such as those disclosed in patents and patent applications U.S. Pat No. 4,367,390, EP 863 145, EP 517 104, EP 570 838, EP 796 851, EP 775 698, EP 878 469, EP 933 376, EP 507 691, EP 507 692, EP 790 243 or EP 944 624, the entire contents of which are incorporated herein by reference; benzophenone derivatives; β,β-diphenylacrylate derivatives; benzotriazole derivatives; benzalmalonate derivatives; benzimidazole derivatives; imidazolines; bisbenzoazolyl derivatives, such as disclosed in Patents EP 669 323 and U.S. Pat. No. 2,463,264, the entire contents of which are incorporated herein by reference; p-aminobenzoic acid (PABA) derivatives; methylenebis(hydroxyphenylbenzotriazole) derivatives, such as disclosed in patents and patent applications U.S. Pat. Nos. 5,237,071, 5,166,355, GB 2,303,549, DE 19 726 184 and EP 893 119, the entire contents of which are incorporated herein by reference; benzoxazole derivatives, such as disclosed in Patent Applications EP 832 642, EP 1 027 883, EP 1 300137 and DE 10 162 844; screening polymers and screening silicones, such as those disclosed in particular in patent application W093/04665, the entire contents of which are incorporated herein by reference; dimers derived from α-alkylstyrene, such as those disclosed in patent application DE 19 855 649, the entire contents of which are incorporated herein by reference; 4,4-diarylbutadienes, such as disclosed in Applications EP 967 200, DE 19 746 654, DE 19 755 649, EP 1 008 586, EP 1 133 980 and EP 1 133 981, the entire contents of which are incorporated herein by reference, and their mixtures.

The organic photoprotective agents more particularly preferred may be chosen from the following compounds (CTFA names or chemical names):
Ethylhexyl Salicylate,
Ethylhexyl Methoxycinnamate,
Octocrylene,
Phenylbenzimidazole Sulfonic Acid,
Benzophenone-3,
Benzophenone-4,
Benzophenone-5,
4-Methylbenzylidene Camphor,
Terephthalylidene Dicamphor Sulfonic Acid,
Disodium Phenyl Dibenzimidazole Tetrasulfonate,
2,4,6-Tris(diisobutyl 4′-aminobenzalmalonate)-s-triazine,
Anisotriazine,
Ethylhexyl Triazone,
Diethylhexyl Butamido Triazone,
Methylene Bis-Benzotriazolyl Tetramethyl-butylphenol,
Drometrizole Trisiloxane,
Polysilicone-15,
1,1-Dicarboxy (2,2′-dimethylpropyl)-4,4-diphenylbutadiene,
2,4-Bis[5-1(dimethylpropyl)benzoxazol-2-yl-(4-phenyl)imino]-6-(2-ethylhexyl)imino-1,3,5-triazine, and their mixtures.

Suitable inorganic photoprotective agents may be chosen from pigments or alternatively nanopigments (mean size of the primary particles: generally between 5 nm and 100 nm, preferably between 10 nm and 50 nm) formed of metal oxides which may or may not be coated, such as, for example, nanopigments formed of titanium oxide (amorphous or crystalline in the rutile and/or anatase form), iron oxide, zinc oxide, zirconium oxide or cerium oxide, which are all UV photoprotective agents well known per se. Furthermore, conventional coating agents are alumina and/or aluminum stearate. Such nanopigments formed of metal oxides, which may or may not be coated, are disclosed in particular in Patent Applications EP 518 772 and EP 518 773, the entire contents of which are incorporated herein by reference.

The photoprotective agents can be present in the photoprotective composition in amounts ranging from 0.1 to 35% by weight, and preferably from 5 to 30% by weight, based on the weight of the photoprotective composition. The photoprotective composition preferably has an SPF value of at least 15, such as 20 or 30 or greater.

In order to enhance soothing and/or to lessen irritation, the skin soothing composition and/or the maintenance composition may be formulated with thermal or mineral waters. By “thermal or mineral water” is meant water having a mineral content of at least 300 mg/l.

In this respect, the term “mineral content” is understood to mean the sum of the concentrations of anions and of cations present in the thermal or mineral water. The fact of using a water with a high mineral content makes it possible to compensate for the irritating effect of the anti-acne and the keratolytic compositions employed previously.

In the present invention, use is made without distinction of a thermal water or of a mineral water. Generally, a mineral water is suitable for consumption, which is not always the case with a thermal water. Each of these waters comprises, inter alia, trace elements and dissolved minerals.

The thermal and/or mineral water used according to the invention can have a mineral content of at least 400 mg/l, in particular of at least 700 mg/l, and more particularly a total concentration of carbonates and of bicarbonates of at least 150 mg/l and more preferably of at least 360 mg/l and in particular of sodium carbonate and bicarbonate of greater than 2 mg/l. The concentration of silicon oxide in the water used in the composition according to the invention can preferably be at least 6 mg/l and more preferably at least 9 mg/l.

The thermal water or the mineral water used according to the invention can be chosen from water from Avène, water from Vittel, waters from the Vichy basin, water from Uriage, water from La Roche Posay, water from La Bourboule, water from Enghien-les-Bains, water from Saint Gervais-les-Bains, water from Néris-les-Bains, water from Allevard-les-Bains, water from Digne, water from Maizières, water from Neyrac-les-Bains, water from Lons-le-Saunier, water from Eaux-Bonnes, water from Rochefort, water from Saint Christau, water from Les Fumades and water from Tercis-les-Bains.

Among these waters, those which exhibit a mineral content of less than 700 mg/l but of greater than 400 mg/l are water from La Roche Posay, water from Eaux-Bonnes or water from Saint Christau.

Among these waters, those which exhibit a total concentration of carbonates or bicarbonates of greater than 360 mg/l are water from Vittel, water from La Bourboule, water from Les Fumades, water from Enghien-les-Bains, water from La Roche Posay, water from the Vichy basin or water from Uriage.

Among these waters, those which exhibit a concentration of carbonates or bicarbonates of between 150 mg/l and 360 mg/l are water from Digne, water from Maizières, water from Rochefort or water from Saint Gervais-les-Bains.

Among these waters, those which comprise at least 2 mg/l of sodium carbonate or bicarbonate are water from La Roche Posay, water from Vittel, waters from the Vichy basin and water from Uriage.

The waters comprising at least 9 mg/l of silicon oxide are water from La Roche Posay, water from Vittel, waters from the Vichy basin or water from Uriage.

Waters from the Vichy basin are preferred for use in the present invention.

The water used in the skin soothing composition and/or the maintenance composition may be water suitable for use in cosmetic or dermatological compositions, which may be exclusively or partially a mineral or thermal water as defined above. The skin soothing composition and/or maintenance composition according to the invention generally may thus comprise no mineral or thermal water, more than 2% by weight, preferably more than 5% by weight, more preferably more than 7% by weight, indeed even make up all the water contained in the skin soothing composition and/or maintenance composition.

The compositions may be provided in all dosage forms conventionally used for topical application and, in particular, in the form of: (i) dispersions of the lotion or gel type, (ii) emulsions with a liquid or semi-liquid consistency of the milk type, obtained by dispersion of a fatty phase in an aqueous phase (O/W) or vice versa (W/Q), (iii) suspensions or emulsions with a soft, semi-solid or solid consistency of the cream or gel type, (iv) multiple emulsions (W/O/W or o/W/0), (v) microemulsions, (vi) vesicular dispersions of ionic and/or nonionic type, or (vii) wax/aqueous phase dispersions. These compositions are prepared according to methods known to those of ordinary skill in the art of cosmetics or dermatological formulations.

The oils present in these emulsions can be silicone oils, which may be volatile or nonvolatile, hydrocarbon oils or vegetable oils. These emulsions can additionally comprise non-oily fatty substances, such as shea butter, silicone gums, esters of fatty acids and of fatty alcohols, fatty acids and fatty alcohols.

These compositions can additionally comprise various adjuvants commonly used in the cosmetics field, such as emulsifiers, including glyceryl fatty acid esters, sugar fatty acid esters, sorbitan fatty acid esters, polyethylene glycol fatty acid esters, ethoxylated fatty alcohols and alkylpolyglycosides; fillers, in particular polyacrylamide (Nylon) fibers and/or microbeads, silica, optionally in the form of a colloidal dispersion, and/or organic microspheres which are optionally expanded; sunscreens; preservatives and/or copreservatives, such as caprylyl glycol; sequestering agents, such as EDTA salts; colorants; fragrances; pH adjusters, such as neutralizing agents and/or buffering agents; ethanol; and thickening and gelling agents, in particular acrylamide homo- and co-polymers, acrylic homo- and co-polymers, acrylamidomethylpropanesulphonic acid (AMPS) homo-and co-polymers, and xanthan gum.

Of course, a person skilled in the art will take care to choose this or these possible additional compounds and/or their amounts so that the advantageous properties of the compositions according to the invention are not, or not substantially, detrimentally affected by the envisaged addition.

A particularly preferred embodiment of the present invention is a cosmetic kit containing:
(a) a unit containing a microdermabrasion composition with 20% by weight, based on the weight of the microdermabrasion composition, of aluminum oxide particles;
(b) a unit containing a skin peeling composition with 10% by weight, based on the weight of the skin peeling composition, of glycolic acid;
(c) a dropper for dispensing the skin peeling composition;
(d) a unit containing an anti-ageing composition, in powder form, with at least 90% by weight, based on the weight of the anti-ageing composition, of ascorbic acid;
(e) a scoop for dispensing the anti-ageing composition;
(f) a mixing cup for blending the skin peeling composition and the anti-ageing composition to form a blended composition;
(g) a brush for applying the blended composition onto a targeted area of skin;
(h) a unit containing a skin soothing composition; and
(i) a unit containing a maintenance composition.

The present invention also provides for a method of treating skin involving the steps of:
(a) providing a unit containing a microdermabrasion composition;
(b) providing a unit containing a skin peeling composition having at least one skin peeling agent;
(c) providing a unit containing an anti-ageing composition having at least one anti-ageing ingredient, in powder form;
(d) providing a unit containing a skin soothing composition having at least one skin soothing agent;
(e) providing a unit containing a photoprotective composition;
(f) contacting the skin with the microdermabrasion composition to form abraded skin;
(g) removing the microdermabrasion composition from the abraded skin;
(h) mixing the skin peeling composition and the anti-ageing composition to form a line/wrinkle treating composition;
(i) contacting the skin with the line/wrinkle treating composition;
(j) applying a layer of skin soothing composition over the line/wrinkle treating composition;
(k) allowing the line/wrinkle treating composition and the skin soothing composition to remain on the skin for a predetermined period of time;
(l) removing the line/wrinkle treating composition and the skin soothing composition from the skin; and
(m) applying a photoprotective composition onto the skin.

The precise period of time which the line/wrinkle treating composition and the skin soothing composition remain on the skin will depend upon a number of variables including, but not limited to, the concentration of actives contained in the compositions, as well as the desired effect being sought. However, in all instances, said period of time will be apparent to those skilled in the art.

According to yet another embodiment of the present invention, there is provided a first modified cosmetic kit wherein the skin peeling composition and anti-ageing composition may be combined to form a single line/wrinkle treating composition. Thus, according to this embodiment, the first modified kit comprises a multi-unit receptacle containing: (a) a microdermabrasion composition; (b) a line/wrinkle treating composition comprising a mixture of at least one skin peeling agent and at least one anti-ageing ingredient; (c) a skin soothing composition; (d) optionally, a maintenance composition; and (e) optionally, a photoprotective composition.

In this embodiment, the maintenance composition contains at least one active chosen from a skin peeling agent and an anti-ageing ingredient wherein either, or both, of which may be present in a concentration lower than that of the line/wrinkle treating composition.

The present invention thus also provides for a method of treating skin using the above-described first modified cosmetic kit. The method involves the steps of:
(a) providing a unit containing a microdermabrasion composition;
(b) providing a unit containing a line/wrinkle treating composition having at least one active chosen from a skin peeling agent and an anti-ageing ingredient;
(c) providing a unit containing a skin soothing composition;
(d) providing a unit containing a photoprotective composition;
(e) contacting the skin with the microdermabrasion composition to form abraded skin;
(f) removing the microdermabrasion composition from the abraded skin;
(g) contacting the skin with the line/wrinkle treating composition;
(h) applying a layer of skin soothing composition over the line/wrinkle treating composition;
(i) allowing the line/wrinkle treating composition and the skin soothing composition to remain on the skin for a predetermined period of time;
(j) removing the line/wrinkle treating composition and the skin soothing composition from the skin; and
(k) applying a photoprotective composition onto the skin.

The precise period of time which the line/wrinkle treating composition and the skin soothing composition remain on the skin will depend upon a number of variables including, but not limited to, the concentration of actives contained in the compositions, as well as the desired effect being sought. However, in all instances, said period of time will be apparent to those skilled in the art.

Yet another embodiment of the present invention provides for a second modified cosmetic kit containing a skin soothing composition combined with at least one active chosen from a skin peeling agent and an anti-ageing ingredient. Thus, according to this embodiment, the kit comprises a multi-unit receptacle containing: (a) a microdermabrasion composition; (b) a multi-benefit skin composition containing at least one skin soothing ingredient and at least one active chosen from a skin peeling agent and an anti-ageing ingredient; (c) optionally, a maintenance composition; and (d) optionally, a photoprotective composition.

In this embodiment, the maintenance composition contains at least one active chosen from a skin peeling agent and an anti-ageing ingredient wherein either, or both, of which may be present in a concentration lower than that of the multi-benefit skin composition.

The present invention also provides for a method of treating skin using the above-described second modified cosmetic kit. The method involves the steps of:
(a) providing a unit containing a microdermabrasion composition;
(b) providing a unit containing a multi-benefit skin composition having at least one skin soothing ingredient and at least one active chosen from a skin peeling agent and an anti-ageing ingredient;
(c) optionally, providing a unit containing a maintenance composition having at least one active chosen from a skin peeling agent and an anti-ageing ingredient;
(d) providing a unit containing a photoprotective composition, followed by, in succession;
(e) contacting the skin with the microdermabrasion composition to form abraded skin;
(f) removing the microdermabrasion composition from the abraded skin;
(g) contacting the skin with the multi-benefit skin composition;
(h) allowing the multi-benefit skin composition to remain on the skin for a predetermined period of time;
(i) removing the multi-benefit skin composition from the skin; and
(j) applying a photoprotective composition onto the skin.

The cosmetic kits and methods of the present invention may be used on either a daily basis or on alternate days depending on the concentration of ingredients contained in the kit and/or the desired skin treating application. A particularly preferred period of use is 21 days. Variations thereof, however, will be apparent to those of ordinary skill of the art.

In order to minimize the irritation potential associated with the use of the cosmetic kit of the present invention, it is preferred to use the maintenance composition by itself, on alternate days, in place of the other components of the cosmetic kit. Thus, in use, a person may apply all of the components of a cosmetic kit with the exception of the maintenance composition on days 1, 3, 5, . . . , and use the maintenance composition by itself on days 2, 4, 6, . . . , over a period of 21 days. Daily use of the photoprotective composition is strongly recommended.

The precise period of time which the multi-benefit skin composition remains on the skin will depend upon a number of variables including, but not limited to, the concentration of actives contained in the compositions, as well as the desired effect being sought. However, in all instances, said period of time will be apparent to those skilled in the art.

The present invention will be better understood from the examples which follow, all of which are intended for illustrative purposes only and are not meant to unduly limit the scope of the invention in any way.

### EXAMPLES

Microdermabrasion Composition

A microdermabrasion composition in accordance with the present invention was prepared having the following formulation:

| Ingredients (CTFA Designation) | % w/w |
|---|---|
| Ammonium Polyacryloyldimethyl Taurate | 0.8000 |
| Glycerin | 5.0000 |
| Preservatives | 1.1000 |
| Butyrospermum Parkii (Shea Butter) | 2.0000 |
| Dimethicone | 2.0000 |
| Glyceryl Stearate/PEG-100 Stearate | 3.0000 |
| Stearyl Alcohol | 1.5000 |
| Mineral Oil | 10.0000 |
| Aluminum oxide | 20.0000 |
| Dye | 0.0005 |
| Water | q.s. 100.000 |

The microdermabrasion composition was applied in the evening to a wetted face, avoiding the outline of the eyes and lips, by massaging with the fingertips for approximately 1 to 2 minutes, followed by rinsing with water and by drying using a clean towel. The use of microdermabrasion composition improved the radiance of the complexion, smoothed the surface of the skin and reduced the appearance of wrinkles. It also prepared the skin for application of the skin peeling composition.

Skin Peeling Composition

A skin peeling composition in accordance with the present invention was prepared having the following formulation:

| Ingredients (CTFA Designation) | % w/w |
|---|---|
| Sodium Hydroxide | 2.60 |
| Propylene Glycol | 0.25 |
| Alcohol Denat. | 5.00 |
| Glycolic Acid | 9.94 |
| Linoleamidopropyl PG-Dimonium-Chloride | 0.30 |
| Phosphate | |
| Hydroxyethylcellulose | 1.50 |
| Water | q.s |
| | 100.00 |

This formula had a pH of from 3.5 to 3.8.

Anti-Ageing Composition

An anti-ageing composition, in powder form, in accordance with the present invention was prepared having the following formulation:

| Ingredients | % w/w |
|---|---|
| Ascorbic Acid | 95.67 |
| Sodium Ascorbate | 0.5 |
| Magnesium Ascorbate/PCA | 0.5 |
| Calcium Ascorbate | 3.7 |
| Sodium Citrate | 0.03 |
| Citric Acid | 0.2 |
| Total | 100 |

A line/wrinkle treating composition was formed, in situ, by mixing 2 scoops of the anti-ageing composition, in powder form (160 mg) with 2 droppers full (1.30 ml) of skin peeling composition, in a mixing cup. The line/wrinkle treating composition was then applied onto the face with a brush.

The line/wrinkle treating composition makes it possible to close the pores, soften wrinkles and fine lines and accelerate skin replacement.

Skin Soothing Composition

A skin soothing composition in accordance with the present invention was prepared having the following formulation:

| Ingredients (CTFA Designation) | % w/w |
|---|---|
| PEG/PPG-18/18 Dimethicone | 1.00 |
| C30-45 Alkyl Dimethicone | 3.00 |
| Preservatives | 1.00 |
| Cyclopentasiloxane | 24.50 |
| Glycerin | 7.00 |
| Propylene Glycol | 3.00 |
| Dimethicone | 1.50 |
| Cetyl Dimethicone | 3.00 |
| Magnesium Sulfate | 1.00 |
| Polysorbate 20 | 0.50 |
| Water | q.s 100.00 |

The pH of the internal aqueous phase was 5.50.

The skin soothing composition was applied over the line/wrinkle treating composition and left on the face overnight.

Maintenance Composition

In order to minimize the irritation potential of the above-described compositions, a maintenance composition was used, on alternate days, in place of the above-described compositions.

A maintenance composition in accordance with the present invention was prepared having the following formulation:

| Ingredients (CTFA Designation) | % w/w |
|---|---|
| PEG/PPG-18/Dimethicone | 1.00 |
| Fragrance | 0.05 |
| Sodium Hydroxide | 1.83 |
| Disodium EDTA | 0.05 |
| Nylon-12 | 2.00 |
| Acrylates Copolymer¹ | 0.50 |
| Ascorbic Acid | 5.00 |
| Cyclopentasiloxane | 19.1o |
| Glycerin | 23.00 |
| Propylene Glycol | 4.00 |
| Citric Acid | 1.24 |
| Preservatives | 0.30 |
| Water | q.s 100 |

¹Expancel ® microspheres

After a period of use spanning 21 days, a noticeable improvement in the cutaneous signs of skin ageing was observed.

What is claimed is:

1. A cosmetic kit for treating human skin comprising a multi-unit receptacle containing:
(a) a unit containing a microdermabrasion composition having at least one type of skin abrading particle;
(b) a unit containing a peeling composition having at least one skin peeling agent;
(c) a unit containing an anti-ageing composition having at least one anti-ageing ingredient;
(d) a unit containing a skin soothing composition having at least one skin soothing ingredient;
(e) optionally, a unit containing a maintenance composition having at least one active chosen from a skin peeling agent and an anti-ageing ingredient and wherein the active is present in the maintenance composition in an amount less than that of the skin peeling and/or anti-ageing compositions; and
(f) optionally, a unit containing a photoprotective composition having an SPF of at least about 15.

2. The kit of claim 1 wherein the anti-ageing composition is ascorbic acid, in powder form.

3. The kit of claim 1 wherein the microdermabrasion composition contains at least one type of skin abrading particle chosen from metal oxides, ground fruit kernels, diatomaceous earth and pumice.

4. The kit of claim 1 wherein the microdermabrasion composition contains aluminum oxide.

5. The kit of claim 1 wherein the at least one skin peeling agent is an α-hydroxy acid.

6. The kit of claim 1 wherein the at least one skin peeling agent is glycolic acid.

7. The kit of claim 1 wherein the at least one anti-ageing ingredient is chosen from ascorbic acid, ascorbic acid derivatives, retinol and retinol derivatives.

8. A method of treating a targeted area of skin comprising:
(a) providing a unit containing a microdermabrasion composition having at least one type of skin abrading particle;
(b) providing a unit containing a skin peeling composition having at least one skin peeling agent;
(c) providing a unit containing an anti-ageing composition having at least one anti-ageing ingredient;
(d) providing a unit containing a skin soothing composition having at least one skin soothing ingredient;
(e) optionally, providing a unit containing a photoprotective composition having an SPF of at least about 15;
(f) contacting the skin with the microdermabrasion composition to form abraded skin;
(g) removing the microdermabrasion composition from the abraded skin;
(h) mixing the skin peeling composition and the anti-ageing composition to form a line/wrinkle treating composition;
(i) contacting the skin with the line/wrinkle treating composition;
(j) applying a layer of skin soothing composition over the line/wrinkle treating composition;
(k) allowing the line/wrinkle treating composition and the skin soothing composition to remain on the skin for a predetermined period of time;
(l) removing the line/wrinkle treating composition and the skin soothing composition from the skin; and
(m) applying a photoprotective composition onto the skin.

9. The method of claim 8 wherein the method is performed daily.

10. The method of claim 8 wherein the method is performed on alternate days.

11. The method of claim 8 wherein the anti-ageing composition is ascorbic acid, in powder form.

12. The method of claim 8 wherein the microdermabrasion composition contains at least one type of skin abrading particle chosen from metal oxides, ground fruit kernels, diatomaceous earth and pumice.

13. The method of claim 8 wherein the microdermabrasion composition contains aluminum oxide.

14. The method of claim 8 wherein the at least one skin peeling agent is an α-hydroxy acid.

15. The method of claim 8 wherein the at least one skin peeling agent is glycolic acid.

16. The method of claim 8 wherein the at least one anti-ageing ingredient is chosen from ascorbic acid, ascorbic acid derivatives, retinol and retinol derivatives.

17. The method of claim 8 further comprising providing a maintenance composition containing at least one active chosen from a skin peeling agent and an anti-ageing ingredient and wherein the active is present in the maintenance composition in an amount less than that of the skin peeling and/or anti-ageing compositions.

18. The method of claim 17 wherein the maintenance composition is applied on the alternate days of claim 10.

19. A cosmetic kit for treating human skin comprising a multi-unit receptacle containing:
(a) a unit containing a microdermabrasion composition having at least one type of skin abrading particle;
(b) a unit containing a line/wrinkle treating composition having a mixture of at least one skin peeling agent and at least one anti-ageing ingredient;
(c) a unit containing a skin soothing composition having at least one skin soothing ingredient;
(d) optionally, a unit containing a maintenance composition having at least one active chosen from a skin peeling agent and an anti-ageing ingredient and wherein the active is present in the maintenance composition in an amount less than that of the skin peeling and/or anti-ageing compositions; and
(e) optionally, a unit containing a photoprotective composition having an SPF of at least about 15.

20. A method of treating a targeted area of skin comprising:
(a) providing a unit containing a microdermabrasion composition having at least one type of skin abrading particle;
(b) providing a unit containing a line/wrinkle treating composition having at least one active chosen from a skin peeling agent and an anti-ageing ingredient;
(c) providing a unit containing a skin soothing composition having at least one skin soothing ingredient;
(d) providing a unit containing a photoprotective composition having an SPF of at least about 15;
(e) contacting the skin with the microdermabrasion composition to form abraded skin;
(f) removing the microdermabrasion composition from the abraded skin;
(g) contacting the skin with the line/wrinkle treating composition;
(h) applying a layer of skin soothing composition over the line/wrinkle treating composition;
(i) allowing the line/wrinkle treating composition and the skin soothing composition to remain on the skin for a predetermined period of time;
(j) removing the line/wrinkle treating composition and the skin soothing composition from the skin; and
(k) applying the photoprotective composition onto the skin.

21. The method of claim 20 further comprising providing a maintenance composition containing at least one active chosen from a skin peeling agent and an anti-ageing ingredient and wherein the active is present in the maintenance composition in an amount less than that of the skin peeling and/or anti-ageing compositions.

22. The method of claim 20 wherein the method is performed daily.

23. The method of claim 20 wherein the method is performed on alternate days.

24. The method of claim 21 wherein the maintenance composition is applied on the alternate days of claim 23.

25. A cosmetic kit for treating human skin comprising a multi-unit receptacle containing:
(a) a microdermabrasion composition having at least one type of skin abrading particle;
(b) a multi-benefit skin composition containing at least one skin soothing ingredient and at least one active chosen from a skin peeling agent and an anti-ageing ingredient;
(c) optionally, a maintenance composition containing at least one active chosen from a skin peeling agent and an anti-ageing ingredient and wherein the active is present in the maintenance composition in an amount less than that of the multi-benefit skin composition; and
(d) optionally, a photoprotective composition having an SPF of at least about 15.

26. A method of treating skin comprising:
(a) providing a unit containing a microdermabrasion composition having at least one type of skin abrading particle;
(b) providing a unit containing a multi-benefit skin composition having at least one skin soothing ingredient and at least one active chosen from a skin peeling agent and an anti-ageing ingredient;
(c) providing a unit containing a photoprotective composition having an SPF of at least about 15;
(d) contacting the skin with the microdermabrasion composition to form abraded skin;
(e) removing the microdermabrasion composition from the abraded skin;
(f) contacting the skin with the multi-benefit skin composition;
(g) allowing the multi-benefit skin composition to remain on the skin for a predetermined period of time;
(h) removing the multi-benefit skin composition from the skin; and
(i) applying the photoprotective composition onto the skin.

27. The method of claim 26 further comprising providing a maintenance composition containing at least one active chosen from a skin peeling agent and an anti-ageing ingredient and wherein the active is present in the maintenance composition in an amount less than that of the multi-benefit skin composition.

28. The method of claim 26 wherein the method is performed daily.

29. The method of claim 26 wherein the method is performed on alternate days.

30. The method of claim 27 wherein the maintenance composition is applied on the alternate days of claim 29.

31. A method of treating skin comprising:
(a) providing a unit containing a microdermabrasion composition;
(b) providing a unit containing a skin peeling composition having at least one skin peeling agent;
(c) providing a unit containing an anti-ageing composition having at least one anti-ageing ingredient, in powder form;
(d) providing a unit containing a skin soothing composition having at least one skin soothing agent;
(e) providing a unit containing a photoprotective composition;
(f) contacting the skin with the microdermabrasion composition to form abraded skin;
(g) removing the microdermabrasion composition from the abraded skin;
(h) mixing the skin peeling composition and the anti-ageing composition to form a line/wrinkle treating composition;
(i) contacting the skin with the line/wrinkle treating composition;
(j) applying a layer of skin soothing composition over the line/wrinkle treating composition;
(k) allowing the line/wrinkle treating composition and the skin soothing composition to remain on the skin for a predetermined period of time;
(l) removing the line/wrinkle treating composition and the skin soothing composition from the skin; and
(m) applying a photoprotective composition onto the skin.

32. A cosmetic kit for treating human skin comprising a multi-unit receptacle containing:
(a) a unit containing a microdermabrasion composition with 20% by weight, based on the weight of the microdermabrasion composition, of aluminum oxide particles;
(b) a unit containing a skin peeling composition with 10% by weight, based on the weight of the skin peeling composition, of glycolic acid;
(c) a dropper for dispensing the skin peeling composition;
(d) a unit containing an anti-ageing composition, in powder form, with at least 90% by weight, based on the weight of the anti-ageing composition, of ascorbic acid;
(e) a scoop for dispensing the anti-ageing composition;
(f) a mixing cup for blending the skin peeling composition and the anti-ageing composition to form a blended composition;
(g) a brush for applying the blended composition onto a targeted area of skin;
(h) a unit containing a skin soothing composition; and
(i) a unit containing a maintenance composition.

33. The kit of claim 32 further comprising at least one water chosen from thermal water and mineral water, and wherein the at least one water is present in the skin soothing composition and/or the maintenance composition.
